(19) United States
(12) Patent Application Publication
(10) Pub. No.: US 2004/0163608 A1
(43) Pub. Date: Aug. 26, 2004

(54) Stable ready-to-use dosage forms containing coloring matter and active chlorine, and methods of making and using same as disinfectants

(76) Inventors: Shimon Freedman, Herzlia (IL); Leon Lipsicas, Ashkelon (IL)

Correspondence Address: Anthony Castorina, G E Ehrlich, Suite 201, 2001 Jefferson Davis Highway, Arlington, VA, 22202, US

(21) Appl. No.: 10/483,410

(22) PCT Filed:
(86) PCT No.: PCT/IL02/00574

(30) Foreign Application Priority Data
Jul. 26, 2001 (IL) 144581

(51) Int. Cl. A01K 29/00

(52) U.S. Cl. 119/652

(57) ABSTRACT

A method of producing a disinfectant or sterilizing liquid, cream, ointment, paste, or gel by dissolving in a solvent a stable ready-to-use dosage form containing coloring matter and an organic chloroxidizer acting as a chlorine donor releasing active chlorine. The coloring matter is in its maximal level of oxidation. The chloroxidizer with coloring matter has a pH of 4-8 and is present in a sufficient quantity to disinfect or sterilize the surface, vessel, or tissue as required while the colored appearance of the liquid, cream, ointment, paste, or gel is stably maintained over a period of time which exceeds the period of time that the disinfectant would be normally used for that purpose.

### FIELD AND BACKGROUND OF THE INVENTION

The present invention relates to stable, ready-to-use dosage forms containing coloring matter and active chlorine. The invention also relates to methods of making such dosage forms and for using them as disinfectants.

Compounds containing (latently) active chlorine are widely used as disinfectants in various medical, veterinary, food and agricultural applications. Since compounds containing chlorine are applied to various surfaces, equipment and biological tissues as disinfectants, it would be highly desirable to color the compounds containing the active chlorine both to serve as a marker that the liquid has been applied to the respective surface as a disinfectant and also to show when the colored disinfecting compound is absent, and further, as a safety measure, to indicate that the chloroxidizer is no longer present in the vehicle. The color (e.g., a dye) is not for the purpose of indicating the level of chlorine available in the compound produced.

Solutions and compounds containing active chlorine are high oxidizing in their chemical behavior. Dyes, stains and pigments can not withstand the oxidizing effect of such chloroxidizing solutions and compounds. Dyes, stains and pigments have their chemical structures altered by oxidation and thus lose their coloring properties. This process also causes a loss in the potency of the chloroxidizer, by quantitatively reducing its oxidizing capacity.

Organic or inorganic soluble and/or insoluble salts that are colored in nature are generally maximally oxidized. Theoretically, therefore, they are stable in a solution with oxidizing properties and accordingly maintain their chemical structure and color even in the presence of such chloroxidizers. This is demonstrated, for example, by the coloration of hypochlorite solutions at a high pH with potassium permanganate (see U.S. Pat. No. 5,685,262). However, in many applications it is desirable to use a colored chloroxidizer for disinfectant purposes.

### OBJECTS AND BRIEF SUMMARY OF THE INVENTION

One object of the present invention is to provide a method of producing a colored disinfecting dosage form, such as a cream, ointment, paste, or gel, of an organic chloroxidizer containing latently available chlorine suitable for disinfectant purposes in which the colored appearance of the product is maintained over a period of time sufficient to complete the disinfecting or sterilizing action, without affecting the stability of the chlorine disinfectant in the product.

Another object of the invention is to provide a method of indicating by color which surface or biological tissue, such as teats of a milking animal, or skin area before a medical procedure, has been treated with a disinfecting product containing active chlorine and coloring matter.

A further object of the invention is to provide a method of demonstrating the absence of the disinfectant after flushing of the liquid with water.

According to one aspect of the present invention, there is provided a method of producing a disinfecting or sterilizing liquid, cream, ointment, paste, or gel by dissolving in a solvent a stable ready-to-use dosage form containing coloring matter and an organic chloroxidizer acting as a chlorine donor releasing active chlorine; the coloring matter being in its maximal level of oxidation; the chloroxidizer with coloring matter having a pH of 4-8 and being present in a sufficient quantity to disinfect or sterilize the surface, vessel, or tissue as required while the colored appearance of the liquid, cream, ointment, paste, or gel is stably maintained over a period of time which exceeds the period of time that the disinfectant would be normally used for that purpose.

The present invention thus differs from the above-cited U.S. Pat. No. 5,685,262 both in the pH range (4-8, i.e. from acidic to slightly alkaline), of the liquid obtained from the dosage form (a solid or semi-solid vs. a liquid), and in the use of an organic chloroxidizer vs. an inorganic hypochlorite solution.

In some formulations, this can be several weeks, even when the Latent Available Chlorine is present in high concentrations, which may be up to 5000 ppm. The period of coloration is variable and can be influenced by changes in formulation.

According to another aspect of the present invention, there is provided a method or producing a colored disinfectant by mixing in a liquid a solid or semi-solid dosage form containing an organic chloroxidizer together with coloring matter which will produce a colored appearance in the resultant product without affecting the stability of the chloroxidizer; the coloring matter being in its maximal level of oxidation and not susceptible to further oxidation; the organic chloroxidizer having a pH of 4-8 and acting as a chlorine donor releasing active chlorine in the liquid containing the coloring matter without affecting the colored appearance of the liquid over a suitable period of time.

According to a still further aspect of the invention, there is provided a method of indicating, by color, which surface or biological tissue, such as teats of a milking animal, or skin area before a medical procedure, has been treated with a disinfecting liquid, cream, ointment, paste, or gel, by applying to the surface or biological tissue a formulation containing an organic chloroxidizer having a pH of 4-8 and acting as a chlorine donor of active chlorine, together with a coloring matter; the coloring matter being in its maximal level of oxidation.

According to yet a further aspect of the invention, there is provided a colored disinfectant liquid, cream, ointment, paste, or gel comprising a solid or semi-solid formulation dissolved in a solvent and containing an organic chloroxidizer acting as a chlorine donor releasing active chlorine, and coloring matter added thereto; the coloring matter being in its maximal level of oxidation; the chloroxidizer with coloring matter having a pH of 4-8 and being present in a sufficient quantity to disinfect a surface, vessel, or tissue as required while the liquid, cream, ointment, paste, or gel stably maintains its colored appearance over a period of time which exceeds the period of time that the liquid would be normally used for that purpose.

In the preferred embodiments of the invention described below, the organic chloroxidizer is Troclosene (dichloro-s-triazinetrione) or its salts; and is present up to 5,000 ppm LAC (latent active chlorine). The coloring matter is in its maximal level of oxidation and is preferably selected from the group of ferric oxide saccharate, Prussian Blue and its derivatives, and titanium oxide; however, other suitable non-oxidisable compounds may be used.

Further features and advantages of the invention will be apparent from the description below.

The invention is herein described, by way of example, with reference to the following examples:

### BASE FORMULATION

The following examples were added to a solid or semi solid Troclosene (dichloro-s-triazinetrione) salt formulation, containing an organic acid and/or an alkaline salt for pH adjustment, which when dissolved in water or suitable aqueous solvent produced a disinfectant/sterilizing liquid with a concentration of between 150 and 3000 ppm L.A.C. (latent available chlorine) and a near neutral pH between 4 to 8.

### EXAMPLE 1

A brown colored liquid is obtained when ferric oxide saccharate is added to the base formulation. The colored liquid obtained when the resultant formulation is dissolved in water or suitable aqueous solvent, remains unchanged for a suitable extended period of time.

### EXAMPLE 2

A compound which may be varied in its hue between blue and green is obtained when chemicals which react stoichiometrically to form the characteristically insoluble Prussian Blue or a derivative thereof are admixed, dried and then added to the base formulation. The colored liquid obtained when the resultant formulation is dissolved in water or other suitable aqueous solvent remains unchanged and homogeneous for at least 24 hours.

### EXAMPLE 3

An opaque white smooth semi-solid preparation is obtained when titanium oxide is added to a formulation containing a Troclosene salt together with inert excipients.

In the foregoing examples, the Troclosene salt is present up to 5,000 ppm LAC, preferably in the range of 150 to 3,000 ppm L.A.C. Screening of the colored examples using this range of biocidal concentrations have not shown less biocidal activity than the base formulation of the same concentration without color.

Following are examples of many of the applications that liquids containing Troclosene monovalent salt, and prepared with coloring matter in accordance with the above-described method for examples 1 and 2, may be used:

In dipping or spraying of the teats of mammals (such as cows, goats, buffalo, sheep, camels, llamas, yaks) prior to or after milking in the prevention of mastitis, wherein the color allows the operator to see which teats have been treated;

In the cleaning of dairy apparatus, including formulations containing other ingredients such as, but not only, surfactants, wherein the color acts as a marker to show when the final rinse is satisfactorily completed;

In the hygiene, disinfecting and sterilization of medical and allied apparatus, wherein a colored liquid is more desirable than a clear, colorless solution;

In the cleaning of apparatus and surfaces used in the food and catering industry, including formulations containing other ingredients such as, but not only, surfactants, wherein the color acts as an indicating marker to highlight areas which are not in contact with the disinfecting liquid and also to show when the final rinse is satisfactorily completed;

In the dipping of plant cuttings in agricultural and horticultural applications, wherein the color allows the operator to see which plants have been treated;

The coloring of products containing organic chloroxidizing compounds, and in particular Troclosene monovalent salt, may also be used in swimming pools, ornamental pools, and similar applications; in the sanitation of water in vases or other containers in which cut flowers are placed in order to prolong their life; and in the coloring of the drinking water of fowls and mammals in order to show that the water was chlorinated.

Following are additional examples of applications containing Troclosene monovalent salt, with the coloring matter in accordance with the above-described methods for all the Examples 1, 2 and 3:

In the antisepsis and cleaning of the skin in humans and animals prior to surgical or similar procedures, wherein the color acts as an indicator to delineate the areas which have been disinfected;

In the antisepsis and cleaning of surfaces which are dark in color, and the white opaqueness of Example 3 enables the highlighting of areas which are not in contact with the disinfecting agent and also to show when the final rinse is satisfactorily completed.

The base formulation may be used in the form of:

a soluble or soluble effervescent tablet;

a measured quantity of soluble or soluble effervescent granules;

a cream, ointment, paste, or gel;

a liquid in a sealed bottle or sachet.

It will also be appreciated that the above-described method may also be used to indicate which surface or biological tissue, such as teats of a milking animal or skin area before a medical procedure, has been treated with the disinfectant liquid, cream, ointment, paste or gel; and also that the absence of the disinfectant after use may be indicated by flushing the surface or tissue with water until the absence of color is noted in the flushed water.

While the invention has been described with respect to several embodiments, it will be appreciated that these are set forth merely for purposes of example, and that many other variations, modifications and applications of the invention may be made.

What is claimed is:

1. A method of producing a disinfectant or sterilizing liquid, cream, ointment, paste, or gel by dissolving in a solvent a stable ready-to-use dosage form containing coloring matter and an organic chloroxidizer acting as a chlorine donor releasing active chlorine; said coloring matter being in its maximal level of oxidation; said chloroxidizer with coloring matter having a pH of 4-8 and being present in a sufficient quantity to disinfect or sterilize the surface, vessel, or tissue as required while the colored appearance of the liquid, cream, ointment, paste, or gel is stably maintained over a period of time which exceeds the period of time that the disinfectant would be normally used for that purpose.

2. The method according to claim 1, wherein the said solid or semi-solid formulation is soluble in an aqueous solvent.

3. The method according to claim 1, wherein said aqueous solvent is water.

4. The method according to claim 1, wherein said chloroxidizer is an organic compound which upon solution in an aqueous liquid releases active chlorine at a sufficient level to be used as a disinfectant.

5. The method according to claim 1, wherein said chloroxidizer is Troclosene (dichloro-s-triazinetrione) or a salt thereof.

6. The method according to claim 1, wherein said organic chloroxidizer is present up to 5,000 ppm LAC (latent active chlorine).

7. The method according to claim 1, wherein said coloring matter is or includes ferric oxide saccharate.

8. The method according to claim 1, wherein said coloring matter is or includes Prussian Blue or a derivative thereof.

9. The method according to claim 1, wherein said coloring matter is or includes titanium oxide.

10. A method of producing a colored disinfectant by mixing in a liquid a solid or semi-solid dosage form containing an organic chloroxidizer together with coloring matter which will produce a colored appearance in the resultant product without affecting the stability of the chloroxidizer; said coloring matter being in its maximal level of oxidation; said organic chloroxidizer having a pH of 4-8 and acting as a chlorine donor releasing active chlorine in the liquid containing the coloring matter without affecting the colored appearance of the liquid over a period of time.

11. The method according to claim 10, wherein the said solid or semi-solid formulation is soluble in said liquid.

12. The method according to claim 10, wherein said liquid is water.

13. The method according to claim 10, wherein said organic chloroxidizer is Troclosene (dichloro-s-triazinetrione) or a salt thereof.

14. The method according to claim 10, wherein said organic chloroxidizer is present up to 5,000 ppm LAC (latent active chlorine).

15. The method according to claim 10, wherein said coloring matter is or includes ferric oxide saccharate.

16. The method according to claim 10, wherein said coloring matter is or includes Prussian Blue or a derivative thereof.

17. The method according to claim 10, wherein said coloring matter is or includes titanium oxide.

18. A method of indicating, by color, which surface or biological tissue, such as teats of a milking animal, or skin area before a medical procedure, has been treated with a disinfecting liquid, cream, ointment, paste, or gel, by applying to said surface or biological tissue a formulation containing an organic chloroxidizer having a pH of 4-8 and acting as a chlorine donor of active chlorine, together with a coloring matter; said coloring matter being in its maximal level of oxidation.

19. A method of indicating the absence of the disinfectant after use according to the method of claim 18, by flushing the surface or tissue with water until the absence of color is noted in the flush water.

20. A method of coloring a disinfectant product, wherein a maximally oxidized substance is used as a coloring matter in conjunction with an organic chlorine donor.

21. A colored disinfectant liquid, cream, ointment, paste, or gel comprising a solid or semi-solid formulation dissolved in a solvent and containing an organic chloroxidizer acting as a chlorine donor releasing active chlorine, and coloring matter added thereto; said coloring matter being in its maximal level of oxidation; said chloroxidizer with coloring matter having a pH of 4-8 and being present in a sufficient quantity to disinfect or sterilize a surface, vessel, or tissue as required while the liquid, cream, ointment, paste, or gel stably maintains its colored appearance over a period of time which exceeds the period of time that the resultant product liquid would normally be used for that purpose.

22. The colored disinfectant according to claim 21, wherein the said solid or semi-solid formulation is soluble in an aqueous solvent.

23. The colored disinfectant according to claim 20, wherein said organic chloroxidizer is Troclosene (dichloro-s-triazinetrione).

24. The colored disinfectant according to claim 20, wherein said organic chloroxidizer is present up to 5,000 ppm LAC (latent active chlorine).

25. The colored disinfectant according to claim 20, wherein said coloring matter is or includes ferric oxide saccharate.

26. The colored disinfectant according to claim 20, wherein said coloring matter is or includes Prussian Blue or a derivative thereof.

27. The colored disinfectant according to claim 20, wherein said coloring matter is or includes titanium oxide.
